(19) United States
(12) Patent Application Publication
(10) Pub. No.: US 2017/0305643 A1
(43) Pub. Date: Oct. 26, 2017

(54) COTTON SWAB STICKS DISPENSER

(71) Applicants: HAGIT ZOHAR MORAN, GANEI TIKVA (IL); ARIK YOSEF SAPIR, RISHON LE ZION (IL)

(72) Inventors: HAGIT ZOHAR MORAN, GANEI TIKVA (IL); ARIK YOSEF SAPIR, RISHON LE ZION (IL)

(21) Appl. No.: 15/490,919

(22) Filed: Apr. 19, 2017

Related U.S. Application Data
(60) Provisional application No. 62/325,479, filed on Apr. 21, 2016.

(51) Int. Cl. B65D 83/02; A61F 13/38

(52) U.S. Cl. CPC B65D 83/02; A61F 13/38

(57) ABSTRACT

A cotton swab sticks dispenser, including at least one track for housing a portion of each of a plurality of cotton swab sticks therewithin, width of the at least one track being the thickness of one rod of the cotton swab sticks, thereby the track locates the cotton swab sticks in a line along the at least one track, a barrier, for limiting a last cotton swab of the cotton swab sticks from advancing along the line, and an outlet of the at least one track, being disposed orthogonally to the line, thereby dispensing of the last cotton swab is applicable by moving thereof orthogonally to the line.

### CROSS-REFERENCE TO RELATED APPLICATIONS

This application claims the benefit of priority from U.S. Provisional Application No. 62/325,479, filed May 9, 2016, the disclosure of which is incorporated herein by reference.

### TECHNICAL FIELD

The invention relates to the field of dispensers. More particularly, the invention relates to a dispenser for cotton swab sticks, since it requires sterilization.

### BACKGROUND

One of the problems regarding cotton swab sticks is in that the cotton wad is thicker than that of the rod. Another problem is that the rod is significantly thin.

There is a long felt need to provide a dispenser for cotton swab sticks, providing access to one cotton swab each time.

### BRIEF DESCRIPTION OF THE DRAWINGS

Embodiments, features, and aspects of the invention are described herein in conjunction with the following drawings:

FIG. 1 is a perspective view of a cotton swab sticks dispenser according to one embodiment of the invention.

FIG. 2 is a top perspective view of the dispensing mechanism separated from the package, both of FIG. 1.

FIG. 3 is a side view of the dispensing mechanism of FIG. 2 prior to the dispensing operation.

FIG. 4 is a side view of the dispensing mechanism of FIG. 2 at the dispensing operation.

FIG. 5 is a cut side perspective view of the dispensing mechanism of FIG. 1.

The drawings are not necessarily drawn to scale.

### DETAILED DESCRIPTION

The invention will be understood from the following detailed description of embodiments of the invention, which are meant to be descriptive and not limiting. For the sake of brevity, some well-known features are not described in detail.

The reference numbers have been used to point out elements in the embodiments described and illustrated herein, in order to facilitate the understanding of the invention. They are meant to be merely illustrative, and not limiting. Also, the foregoing embodiments of the invention have been described and illustrated in conjunction with systems and methods thereof, which are meant to be merely illustrative, and not limiting.

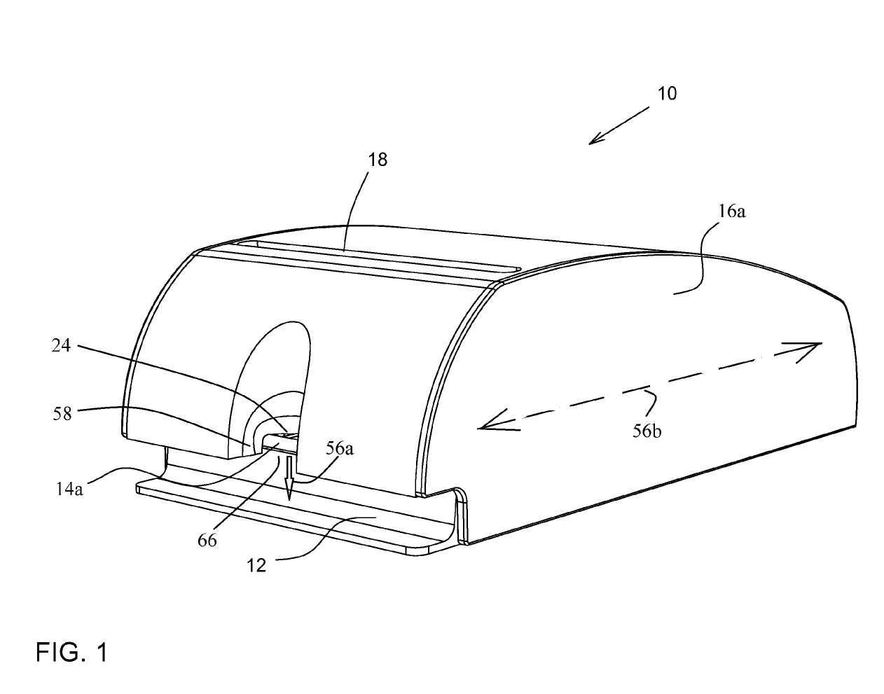

FIG. 1 is a perspective view of a cotton swab sticks dispenser according to one embodiment of the invention.

A cotton swab sticks dispenser 10 includes, according to one embodiment of the invention, a package 16a, and a dispensing mechanism 24 (most of it hidden), for providing access at the outlet 66 to one cotton swab 14a only, at the center of cotton swab 14a, being at the center of the rod of the cotton swab.

Dispensing mechanism 24 presses perpendicularly to cotton swab 14a thereon to the direction shown by an arrow 56a. However, a barrier 58 of package 16a limits cotton swab 14a from advancing along direction 56a.

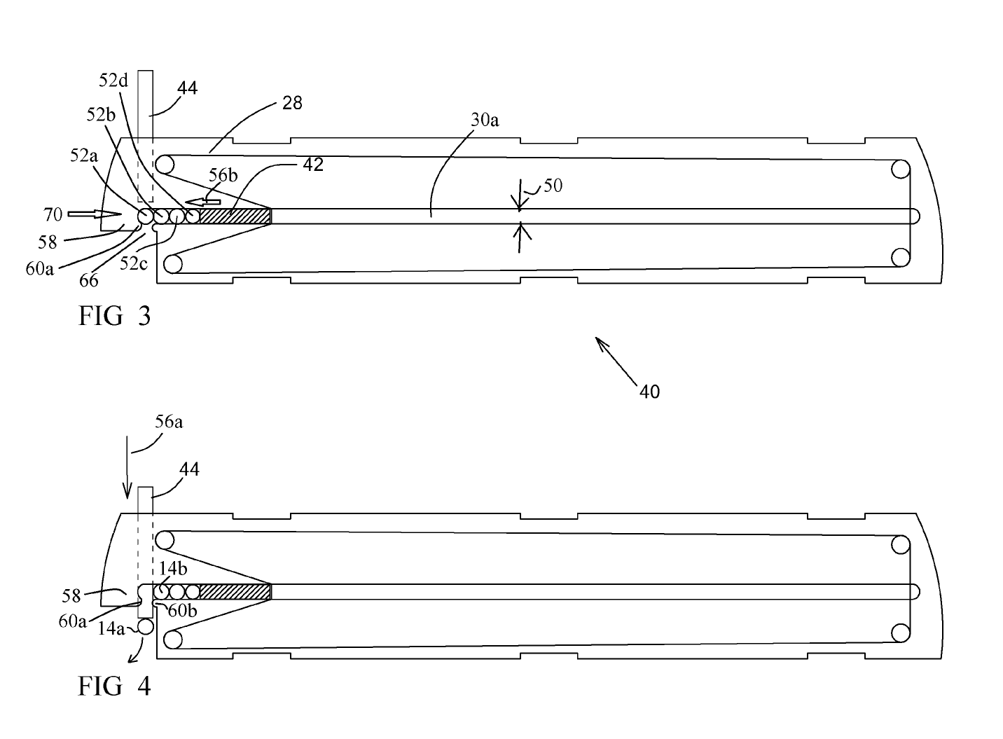

Dispensing of cotton swab 14a is obtained by manually pulling or pressing cotton swab 14a downwards, either by a finger, or by a dispensing button 44 (shown in FIG. 3), as shown by an arrow 56a. Direction 56a is orthogonal to cotton swab 14 and to the advancing direction 56b.

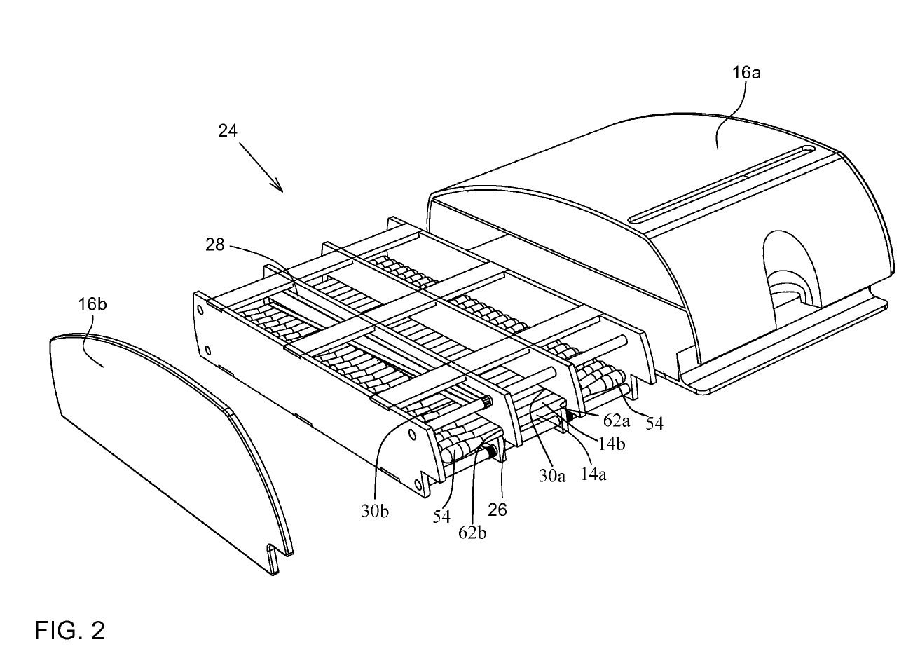

Upon dispensing cotton swab 14a, a new cotton swab 14b (shown in FIG. 2) will be presented instead of cotton swab 14a.

FIG. 2 is a top perspective view of the dispensing mechanism separated from the package, both of FIG. 1.

Dispensing mechanism 24, according to one embodiment includes tracks 30a and 30b, being elongated or at least having elongated portions. each for directing a plurality of cotton swab sticks 14a, 14b, etc.

Each of cotton swab sticks 14a, 14b, etc. is directed both by track 30a and 30b, as each of tracks 30a and 30b directs a different portion of the length of the cotton swab, thus together leading together at the correct angle of disposition of the swab in relation to the track. Namely, track 30a directs portion 62a of cotton swab 14a, and track 30b directs portion 62b of cotton swab 14a.

Thus, small portions 62a and 62B only are housed and directed by tracks 30a and 30b, whereas the cotton wads 54, disposed at the ends of cotton swab 14a, are not housed in the tracks.

FIG. 3 is a side view of the dispensing mechanism of FIG. 2 prior to the dispensing operation.

The width 50 of track 30a is substantially the thickness (diameter) of each of rods 52a, 52b, etc. thus pressure applied on a last rod 52d, by a pressing object 42, travelling as well within track 30, provides a neat motion of rods 52a, 52b, 52c, and 52d in a line, as the rods must travel one after and adjacent to the other in a line 70.

An elastic band 28 may press pressing object 42 onto rod 52d.

FIG. 4 is a side view of the dispensing mechanism of FIG. 2 at the dispensing operation.

Pressing on dispensing button 44 along direction 56a presses cotton swab 14a, thereby dispenses it.

Protrusion 60a extending from barrier 58, and protrusion 60b extending from the other side, restrain the dispensing, such that even in case dispensing button 44 slightly presses an edge of cotton swab 14b, protrusions 60a and 60b will disturb dispensing of cotton swab 14b.

Consequently, pressing object 42 presses, through other cotton swab sticks, cotton swab 14b to barrier 58, and any of protrusions 60a and 60b hinder dispensing thereof.

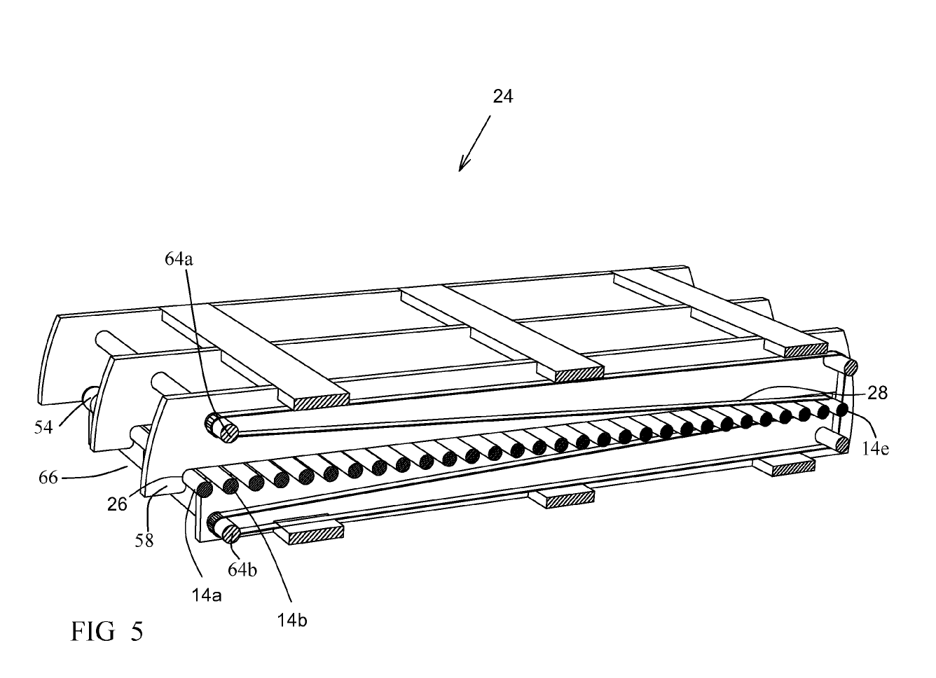

FIG. 5 is a cut side perspective view of the dispensing mechanism of FIG. 1.

Elastic band 28 is anchored to a top anchor 64a and to a bottom anchor 64b, being disposed near outlet 66, and extending to pressing object 42 of FIG. 3 or to the last cotton swab 14e onto rod 52d.

Thus, in one aspect, the invention is directed to a cotton swab sticks (14a, 14B) dispenser (10), including:
at least one track (30a) for housing a portion (62a) of each of a plurality of cotton swab sticks (14a, 14B) therewithin, width (50) of the at least one track (30a) being the thickness of one rod (52a) of the cotton swab sticks (14a, 14B), thereby the track locates the cotton swab sticks (14a, 14B) in a line (70) along the at least one track (30a);
a barrier (58), for limiting a last cotton swab (14a) of the cotton swab sticks (14a, 14B) from advancing along the line (70); and
an outlet (66) of the at least one track (30a), being disposed orthogonally to the line (70),
   thereby dispensing of the last cotton swab (14a) is applicable by moving thereof orthogonally (56a) to the line (70). The barrier and the change of the direction makes the dispensing reliable for dispensing one cotton swab only at a time.

The cotton swab sticks (14a, 14B) dispenser (10) may further include:
a dispensing button (44), for manually pressing thereon, for pressing on the last cotton swab (14a) orthogonally (56a) to the line (70).

The cotton swab sticks (14a, 14B) dispenser (10) may further include:
at least one protrusion (60a, 60b), extending from the barrier (58), for temporally restricting the size of the outlet (66), thereby limiting the dispensing of the last cotton swab (14a),
   thereby avoiding dispensing any cotton swab rather the last cotton swab (14a).

The at least one track (30a) may include at least two tracks (30a, 30b), thereby disposing each of the cotton swab sticks (14a, 14B) orthogonally to the at least two tracks (30a, 30b), since two tracks determine a line of the length of the rod.

The portion (62a, 62b) of each of the plurality of cotton swab sticks (14a, 14B) housed by the at least one track (30a) does not include the cotton wads (54) disposed at ends of the cotton swab sticks (14a, 14b).

The cotton swab sticks (14a, 14B) dispenser (10) may further include:
springy means (28), for pressing the first cotton swab (14e) of the line (70) of the cotton swab sticks (14a, 14B), thereby the locating of the cotton swab sticks (14a, 14B) in the line (70) induces each of the cotton swab sticks (14b) to press the cotton swab (14a) adjacent thereto along the line (70);

The springy means (28) may include:
an elastic band (28), being anchored to a top anchor (64a) and to a bottom anchor (64b) and extending to the first cotton swab (14e),
   wherein the anchors are disposed near the outlet (66),
   thereby increasing an elastic range of the springy means (28).

In the figures and/or description herein, the following reference numerals (Reference Signs List) have been mentioned:
numeral 10 denotes the cotton swab sticks dispenser according to one embodiment of the invention;
numeral 12 denotes a tray for receiving the dispensed cotton swab;
numerals 14a, 14b and 14e denote cotton swab sticks;
numeral 16a denotes the package;
numeral 16b denotes the package cover;
numeral 18 denotes a trash tray;
numeral 24 denotes the dispensing mechanism;
numeral 28 denotes the elastic band or any other springy means;
numerals 30a and 30b denote tracks;
numeral 42 denotes the pressing object;
numeral 44 denotes the dispensing button;
numeral 50 denotes the width of the track;
numerals 52a, 52b, 52c and 52d denote rods, each of a different cotton wad, namely rod 52a is of cotton swab 14a, and rod 52b is of cotton swab 14b, etc.;
numeral 54 denotes a cotton swab;
numeral 56a denotes the direction of the manual dispensing;
numeral 56b denotes the direction of the pressing by the elastic band or any other springy means;
numeral 58 denotes the barrier;
numerals 60a and 60b denote protrusions, for restricting the size of the outlet;
numerals 62a and 62b denote portions of the rod being housed and directed by the tracks;
numerals 64a and 64b denote top and bottom anchors respectively;
numeral 66 denotes the outlet, where the cotton swab sticks are dispensed; and
numeral 70 denotes the line, in which the cotton swab sticks are placed within the track.

The foregoing description and illustrations of the embodiments of the invention has been presented for the purposes of illustration. It is not intended to be exhaustive or to limit the invention to the above description in any form.

Any term that has been defined above and used in the claims, should to be interpreted according to this definition.

The reference numbers in the claims are not a part of the claims, but rather used for facilitating the reading thereof. These reference numbers should not be interpreted as limiting the claims in any form.

What is claimed is:

1. A cotton swab sticks dispenser, comprising:
at least one track for housing a portion of each of a plurality of cotton swab sticks therewithin, width of said at least one track being a thickness of one rod of said cotton swab sticks, thereby locating said cotton swab sticks in a line along said at least one track;
a barrier, for limiting a last cotton swab of said cotton swab sticks from advancing along said line; and
an outlet of said at least one track, being disposed orthogonally to said line,
thereby dispensing of said last cotton swab is applicable by moving thereof orthogonally to said line.

2. A cotton swab sticks dispenser according to claim 1, further comprising:
a dispensing button, for manually pressing thereon, for pressing on said last cotton swab orthogonally to said line.

3. A cotton swab sticks dispenser according to claim 1, further comprising:
at least one protrusion, extending from said barrier, for temporally restricting a size of said outlet, thereby limiting said dispensing of said last cotton swab,
thereby avoiding dispensing any cotton swab rather said last cotton swab.

4. A cotton swab sticks dispenser according to claim 1, wherein said at least one track comprises at least two tracks, thereby disposing each of said cotton swab sticks orthogonally to said at least two tracks.

5. A cotton swab sticks dispenser according to claim 1, wherein said portion of each of said plurality of cotton swab sticks housed by said at least one track does not comprise cotton wads disposed at ends of said cotton swab sticks.

6. A cotton swab sticks dispenser according to claim 1, further comprising:
springy means, for pressing a first cotton swab of said line of said cotton swab sticks, thereby said locating of said cotton swab sticks in said line induces each of said cotton swab sticks to press a cotton swab adjacent thereto along said line.

7. A cotton swab sticks dispenser according to claim 6, wherein said springy means comprises:
an elastic band, being anchored to a top anchor and to a bottom anchor and extending to said first cotton swab,
wherein said anchors are disposed near said outlet,
thereby increasing an elastic range of said springy means.
